(19) United States
(12) Patent Application Publication
(10) Pub. No.: US 2026/0325186 A1
(43) Pub. Date: Oct. 22, 2026

(54) IMPLANT TEMPLATE AND IMPLANT TEMPLATE SYSTEM

(71) Applicant: Aesculap AG, Tuttlingen (DE)

(72) Inventors: Stephan Lindner, Wurmlingen (DE); Andreas Stern, Moenchweiler (DE); Linda Hauschel, Schoerzingen (DE)

(21) Appl. No.: 19/703,457

(22) Filed: Jun. 10, 2026

Related U.S. Application Data
(63) Continuation of application No. PCT/EP2024/085539, filed on Dec. 10, 2024.

(30) Foreign Application Priority Data
Dec. 11, 2023 (DE) 10 2023 134 668.7

(51) Int. Cl. A61B 17/80; A61B 17/00

(52) U.S. Cl. CPC A61B 17/808; A61B 2017/00907

(57) ABSTRACT

An implant template includes a carrier element having a front side and a rear side. The carrier element, which is formed from a flat material, carries or has at least one two-dimensional implant view of an implant. The carrier element also has at least one see-through region, which makes it possible to see through the carrier element. An implant template system can include at least two implant templates that differ from one another.

### CROSS-REFERENCE TO RELATED APPLICATIONS

This application is a continuation of International Application No. PCT/EP 2024/085539, filed on Dec. 10, 2024, and claims priority to German Application No. 10 2023 134 668.7, filed on Dec. 11, 2023. The contents of International Application No. PCT/EP 2024/085539 and German Application No. 10 2023 134 668.7 are incorporated by reference herein in their entireties.

### FIELD

The present disclosure relates to an implant template and to an implant template system comprising at least two implant templates.

### BACKGROUND

Before the implantation of an implant in the body of a human or animal patient, it is known, in order to determine in particular a shape and/or size of the implant to be inserted, to bring one or more trial implants up to the location of the body at which the actual implant is to be fixed. In particular, it is typical to place the trial implant that corresponds in shape and size to the implant to finally be implanted at exactly the location where the implant is to finally be positioned. Size determinations are necessary for virtually all types of implants, be it implants that are fixed to the spine as a replacement for vertebral bodies or intervertebral discs or also for knee or hip joint prostheses.

In order to determine the optimal size and shape adapted to the patient's physiology in the manner described, trial implants must be provided in different shapes and/or sizes. These are complex and therefore costly to produce. In addition, they must be provided sterile for size and shape determination on the patient.

DE 10 2006 031 808 A1 discloses a method for determining and setting the drilling channel for a dental implant and a drilling template for performing the method. A simplified size measurement device is described in DE 60 2004 011 420 T2. A template system for the spine and a method for using the system are disclosed in US 2010/0152781 A1.

### SUMMARY

It is therefore, in particular, an object of the present disclosure to provide a template and a template system with which an implant suitable for the treatment of the patient can be determined in a simple manner.

This object is achieved, in accordance with the present disclosure, by an implant template, which comprises a carrier element having a front side and a rear side, wherein the carrier element is formed from a flat material, wherein the carrier element carries or has at least one two-dimensional implant view of an implant, and wherein the carrier element has at least one see-through region, which makes it possible to see through the carrier element.

With such an implant template, it can be determined in a simple manner what size and/or shape an implant must have in order to fulfill the desired function in the body of a patient. Such an implant template can be positioned in the body exactly at or near the implantation position where the implant is to finally be inserted. The two-dimensional implant view, for example a side view or a top view of the implant, can thus be directly compared with the patient's physiological situation. The at least one see-through region makes it possible, in particular, to keep the surgical site and thus the physiological situation in the body of the patient clearly visible to a user so that they can decide whether the implant, the two-dimensional implant view of which is depicted on the carrier element, is suitable in shape and/or size for the planned treatment of the patient, thus is suitable for implantation. Such an implant template, in particular, can be formed in a simple and cost-effective manner, since a flat material is used as a carrier element. It is therefore not necessary to create a three-dimensional structure, for example in the form of known trial implants, which completely corresponds to the final implant to be implanted. Rather, only a template with at least one, in particular only one single, two-dimensional view of the implant to be implanted is provided in order to determine the optimal size thereof for the patient, namely by comparison with the implant view depicted on the implant template. Therefore, for the determination of the size and/or shape of the implant to be implanted, it is not yet absolutely necessary to finally prepare the surgical site for the implantation, because the implant template does not have to be brought into the final implantation position. Rather, it can simply be brought up to or placed against body structures of the patient, for example bone structures. In particular, a proposed implant template can also be handled in a simple manner. In addition, the optimal size and/or shape of the implant to be implanted can be determined in particular in a simple manner by providing a plurality of such implant templates on which implant views of implants of different shapes and/or sizes are depicted, and by comparing the physiological situation in the body of the patient with a plurality of such implant templates. In addition, due to being formed from a flat material, the implant template can be introduced into a patient's body in particular in a quick and simple manner, namely through an opening that is typically smaller than that required for the insertion of the implant to be implanted. In one embodiment, the carrier element may in particular be larger than the two-dimensional implant view. Thus, for example, one or more holding regions for handling the implant template can be formed.

The at least one see-through region can be formed in a simple manner if it is in the form of a perforation or a translucent or transparent carrier element region of the carrier element. Both a perforation and a transparent carrier element region make it possible to see directly through, in particular to body structures behind the implant template. Thus, for example, distances between adjacent vertebral bodies on a spine of a patent can be viewed and also measured through such see-through regions. A translucent carrier element region in this sense is partially light-transmissive. Unlike complete light transmittance, referred to as transparent in the sense of this application, a translucent carrier element region is understood to be semi-transparent. In the following, the terms image-transmissive or see-through or clear are used as synonyms for transparent. Translucent elements commonly used in everyday life are known, for example, in the form of frosted glass panes or matted panes made of glass or plastic, which prevent a direct view through them, but allow light to pass through. Translucent carrier element regions, however, also allow the recognition of structures located behind them, namely when such a carrier element region is brought close enough to the respective body structure of the patient. In particular, the formation of translucent carrier element regions for the use of the implant template can predetermine that a measurement or a comparison of the two-dimensional implant view on the carrier element with a physiological structure is accurate and easily possible only when the implant template is positioned close enough to the physiological structure, in particular abutting against it. Thus, a measurement of physiological structures can be made with particularly high accuracy, since practically no parallax errors can occur. One or more perforations of the carrier element can be used in particular as holding or handling elements for holding or handling the implant template. This is advantageous in particular when a contour or width of the implant template corresponds to the width or contour of the two-dimensional implant view. In this embodiment, there are then no holding tabs or holding regions projecting from the carrier element over the implant view for handling the implant template. This may be the case, for example, with an implant in the form of a cervical plate. For example, it is possible to engage into suitable perforations of the implant template with a handling instrument having at least one handling member, for example forceps with their two free ends, which thus form two handling members, and to securely handle the implant in this way, thus without holding tabs.

Moreover, it may be favorable if the implant view is at least partially opaque to X-ray. This enables a user, in particular, to see the implant view in relation to a bone of the patient, even under X-ray control, i.e., in particular if they do not have a direct line of sight to the implant template. In particular, the implant view or markings that define it can be designed to be visible during X-ray. This can be achieved, for example, by a metallic color for the application of the two-dimensional implant view or by forming the implant template from a carrier element that is opaque X-ray or at least partially opaque to X-ray, which is produced by etching or waterjet machining. In particular, the flat material from which the carrier element is formed may be transparent to X-ray, but may be provided with a coating that is opaque to X-ray. To display the two-dimensional implant view, the coating can then be removed accordingly, for example by etching or machining with a water jet. Preferably, one or more implant perforations, for example screw holes on the depicted implant, are made visible in the implant view in the X-ray, for example by enrichment or reduction of X-ray opaque material from which the implant template is formed.

It is advantageous if the implant view defines an inner and/or outer contour and if the at least one see-through region is arranged or formed adjoining the inner and/or outer contour. This configuration makes it possible, in particular, by comparing the inner and/or outer contour with the physiological situation in the body of the patient, to optimally determine the size and/or shape of the implant to be implanted.

It is favorable if the implant view comprises at least one implant perforation of the implant. This does not necessarily mean that the carrier element must also have a perforation in the region of the implant perforation. Also, no see-through region has to be formed in the region of the at least one implant perforation. However, it is advantageous if a perforation of the carrier element or an otherwise designed see-through region is formed or provided in the region of the implant perforation. In particular, inner contours of the implant view can thus be used when performing a measurement on the body of the patient.

Preferably, the at least one implant perforation forms the at least one see-through region. This makes it possible, in particular, to view a physiological situation in the body of the patient through the implant template and, if necessary, to perform a measurement in the region of the at least one implant perforation.

The at least one implant perforation favorably comprises a screw hole or a fixation hole. The at least one implant perforation depicted in the two-dimensional implant view on the carrier element serves to receive a screw or another fastening element in the implant to be permanently or finally implanted. This embodiment makes it possible, in particular, to check whether the implant depicted on the carrier element is even suitable to be fixed in the body of the patient. In particular, it can thus be checked whether sufficient bone substance is present in the region of the implant perforation to anchor a fastening element.

The implant template can be formed in a simple and cost-effective manner if the carrier element is of plate-shaped or disk-shaped configuration. The carrier element formed from the flat material may in principle be formed in any shape, for example round, oval, polygonal, in particular rectangular or trapezoidal. The carrier element may, in particular, have a shape that corresponds to the two-dimensional implant view or is geometrically similar to it.

In accordance with a preferred embodiment, provision may be made that the carrier element has a thickness, which is defined by a distance between the front side and the rear side, and that the thickness has a value of at most 3 mm. In particular, the thickness has a value of at most 1 mm. Carrier elements having a thickness in the specified range can, in particular, be provided to be dimensionally-stable, such that the implant template is not deformed in an undesirable way when placed against a bodily structure of the patient, which would make the measurement of body structures more difficult.

The thickness is preferably at least 0.05 mm. In particular, it may be at least 0.5 mm. It is thus also possible, in particular, to form a carrier element from a thin film. A dimensional stability of the carrier is not absolutely necessary.

In order to minimize the risk of rejection reactions in the body of the patient, it is advantageous if the implant template is made of one or more biocompatible materials. For example, the carrier element may be made of a first biocompatible material. The two-dimensional implant view may be made of a further biocompatible material, for example a suitable biocompatible printing ink or a biocompatible coating material to apply the implant view to the carrier element. The two-dimensional implant view may also be made of a metallic flat material and be applied to the carrier element.

Favorably, the carrier element is made of a biocompatible carrier element material. In particular, it can thus be prevented that undesired rejection reactions occur in the body of the patient when the carrier element comes into contact with body tissue.

The implant template can be formed in a simple and cost-effective manner if the carrier element is made of a plastic material. In particular, it may be made of polymethyl methacrylate (PMMA), polyethylene terephthalate glycol (PETG), and/or polypropylene (PP).

The carrier element is preferably made of a transparent or translucent carrier element material. This configuration has the advantage, in particular, that a see-through region is automatically formed everywhere on the carrier element where no two-dimensional implant view is applied or arranged. The two-dimensional implant view may be configured, in particular, opaque, so that it is impermeable in particular to electromagnetic radiation in the visible spectral range and/or to X-rays. The two-dimensional implant view, when brought up to a region of bodily structures, thus in particular covers said region, which corresponds exactly to the size and/or shape of the implant to be implanted in the depicted two-dimensional implant view.

For optimal handling and measurement of bodily structures for determining the size and/or shape of an implant to be implanted, it is advantageous if the implant view depicts a top view of a side of the implant. Preferably, one sees exactly this implant view on the implant template that is visible of the implant after implantation.

The implant template can be formed in a simple and cost-effective manner if the implant view is printed or engraved on the carrier element or is formed by laser marking or by a thin metallic stamped part. For example, the implant view may be formed on the front side of the carrier element. Optionally, it may alternatively or additionally also be formed on the rear side of the carrier element. However, this is not absolutely necessary. It is sufficient to provide the two-dimensional implant view on the front side or the rear side.

It is advantageous if the rear side of the carrier element is translucently matted. This configuration gives the carrier element the translucent property already described above. If the implant template is not brought close enough to a body structure, a user of the implant template cannot recognize the body structure through the carrier element, or only very diffusely. The body structure becomes sufficiently visible only when the carrier element is brought close enough to the body structure, in particular abutting against it. The carrier element can, for example, first be formed from a transparent carrier element material before a matting of the rear side to make it translucent. In particular, this also makes it possible to make only certain partial regions of the carrier element translucent. In particular, see-through regions can thus be created that are transparent, but also see-through regions that are translucent.

In order to be able to place the implant template, in particular, against non-planar body structures, too, it is favorable if the carrier element is configured to be flexibly deformable. This allows it to be optimally placed against one-or multi-dimensionally curved body structures in order to measure them.

Preferably, the carrier element is of curved configuration corresponding to the depicted implant. Such a carrier element is then still formed from a flat material. However, it is deformed according to the depicted implant in order to thus enable an optimal abutment against non-planar body structures.

In order to be able to adapt the implant template individually to non-planar body structures, it is advantageous if the carrier element is of plastically deformable configuration. This has the advantage, in particular, that not only the deformation of the carrier element is possible, but that the carrier element also retains the shape given by deformation, in particular bending, in particular until in particular the function of the implant template is fulfilled, namely to measure a body structure.

For the handling of the implant template, in particular, it is advantageous if it comprises at least one holding element for holding it. In particular, the at least one holding element is configured to be held with a holding instrument. For example, forceps or needle holders can be used as holding instruments. For this purpose, it is advantageous if the at least one holding element is arranged or formed outside the carrier element covered by the two-dimensional implant view. The implant template can thus be held without having a negative influence on the measuring of a body structure of a patient with the implant template.

The at least one holding element is favorably configured in the form of a carrier element region without a two-dimensional implant view. Thus, the implant template can be gripped and held on the carrier element region without an implant view in order to place it against a body structure of a patient. In particular, with a holding instrument, the implant view is thereby not obscured.

For the handling of the implant template, it is advantageous if the at least one holding element is configured projecting from the carrier element. In particular, it may be configured projecting in a plane that is defined by the carrier element in a starting position before the implant template is, for example, deformed. In particular, it may be the plane defined by the undeformed flat material from which the carrier element is formed.

Preferably, the at least one holding element is formed in one piece with the carrier element. In particular, it is formed from the flat material. For example, the carrier element with the holding element can be cut out or punched out of a flat material.

The implant template can be handled in a simple and secure manner if the at least one holding element is configured in the form of a tab projecting from the carrier element. Such a tab can basically have any shape. In particular, it may be of polygonal, for example rectangular or square, configuration. It may also be of semi-circular or semi-oval configuration.

In order to further improve the handling of the implant template, it is advantageous if the at least one holding element is configured to be deformable, in particular bendable, relative to the carrier element. Thus, for example, the at least one holding element can be bent relative to the carrier element if they are formed in one piece from a flat material. This enables a user, in particular, to hold the bent holding element, which forms a holding element plane transverse to the plane defined by the carrier element, with a corresponding holding instrument in a simple and reliable manner. Such a holding element, for example in the form of a holding tab, may also be advantageous in order to be able to evaluate certain position points in a lateral X-ray view, for example screw holes on the depicted implant-even with screw angle. For example, the holding element may be made of a metallic material and be bent in the region of the screw holes of the implant and in an angle, which corresponds to a possible screw entry angle in the respective screw holes on the depicted implant.

It is advantageous if the at least one holding element and the carrier element in a handling position enclose a holding angle and if the holding angle is in a range of 0° to about 120°. In particular, it may be about 90°. For example, a user can bend the holding element relative to the carrier element such that they can bring the implant template with one hand or, for example, a holding instrument up to a body structure, for example one or more bones, in the desired manner.

It is further favorable if the carrier element has at least one bending region, which enables a deformation of the carrier element, and if the at least one holding element is arranged or formed outside the bending region. In this way, it can be ensured, in particular, that the at least one holding element cannot negatively influence a property of the carrier element for deforming the latter, in particular for bending it.

In accordance with a further preferred embodiment, provision may be made that the carrier element has or comprises at least one further piece of implant-specific information. This allows a user not only to recognize the two-dimensional implant view and to use it for measurement purposes, but also, for example, information regarding size, implant type, or the like.

It is advantageous if the implant-specific information is or comprises a size, a length, and/or an article number of the implant depicted. A user can then, for example, directly read all implant-specific information or at least a portion of it on the implant template when they have identified the appropriate implant with the implant template. Thus, no additional brochures, product information, or the like are required to select the desired implant for implantation after using the implant template.

The carrier element favorably carries or comprises at least one measuring scale. In particular, distances of body structures of a patient relative to one another can thus be directly measured or assessed with the implant template. A measuring scale can also be used to assess or precisely determine an orientation of the implant in the body. For example in the case of a spinal implant, distances of the implant from an adjacent intervertebral disc can be checked and assessed accordingly, and under certain circumstances even exactly measured.

It is advantageous if the measuring scale is configured in the form of a millimeter scale. Thus, a distance between bodily structures of a patient can be assessed and, if necessary, measured by a user with sufficient accuracy.

It is favorable if the at least one holding element is arranged or formed adjoining the measuring scale or in the vicinity thereof, in particular in an extension thereof. In particular, the at least one holding element can thus be arranged in such a way that it does not negatively affect a property of the carrier element, namely the property of being able to be bent.

In order to not introduce germs into a patient's body when using the implant template, it is favorable if the implant template is configured to be sterilizable. In particular, it may be configured to be sterilizable by exposure to a gas or electromagnetic radiation or particle radiation or superheated steam. A sterilizable implant template can, in particular, be provided sterile for an application, for example in a sterile packaging, which can then be opened by a user in a sterile environment, for example an operating room.

In accordance with a further preferred embodiment, provision may be made that the implant template has a protective layer and that the protective layer completely covers information applied to the carrier element, in particular the at least one implant view and optional further information. In particular, a protective layer can protect markings, for example printed information. The applied information can either be applied to the carrier element in a scratch-resistant manner or can be protected by such a protective layer in order to not introduce abrasion of the information, for example paint particles from which the information is formed, into the patient's body.

The protective layer can be formed in a simple and cost-effective manner if it is configured in the form of a transparent or translucent adhesive strip. The adhesive strip may, for example, be configured to be adhesive on one side.

For handling the carrier element, it is advantageous if it is of circular, oval or polygonal, in particular triangular, quadrangular, pentagonal, or hexagonal configuration. The shape of the carrier element may thus, in particular, deviate from an outer contour of the two-dimensional implant view, thus the carrier element may protrude beyond the two-dimensional implant view on one or more sides, such that the regions of the carrier element not covered by the two-dimensional implant view are able to be used as a see-through region or holding region.

In order to avoid injury of body tissue with the implant template, it is favorable if edges of the carrier element are of rounded configuration or have a chamfer.

In order to be able to perform an implant determination as quickly as possible, it is favorable if the carrier element has two different implant views. In particular, these may be implant views that depict top views of the implant to be implanted from different directions. Thus, the implant template can be brought in particular from different directions up to a body structure and a size and/or shape can be determined accordingly. Only one single implant template is required for this purpose. Thus, a user does not have to bring two implant templates one after the other up to the body structure, but can instead bring one single implant template in different orientations up to a body structure and hold it there to measure said body structure. In particular, the carrier element can thus be deformed in such a way that the two different implant views define different planes, in particular planes extending transversely, for example perpendicularly, to one another.

Preferably, the two different implant views depict side views of the implant oriented transversely, in particular perpendicularly, to one another. For example, in the case of intervertebral disc implants, intervertebral bodies, femoral components or the like, significantly different implant views can be displayed on one single implant template.

Implant templates can be provided for a wide range of applications. To this end, it is favorable if the at least one implant depicted on the implant template is a spinal implant, a bone plate, a bone screw, an intervertebral disc implant, an intervertebral implant, a knee joint implant, a tibial plate of a knee joint implant, or a femoral component of a knee joint implant. In principle, however, all other implants not mentioned in this non-exhaustive list that are intended for use in the body of a patient can also be applied as two-dimensional implant views on a carrier element for the formation of an implant template.

Preferably, the implant template is configured as a single-use article. This means that there is no longer any need for complex processing, in particular cleaning and sterilization, in order to be used again. For example, it may be made of a biodegradable carrier element material, so that the implant template can be disposed of in an environmentally friendly manner after a single use.

Furthermore, the object stated at the outset is achieved, in accordance with the present disclosure, by an implant template system, which comprises at least two of the implant templates described above, wherein the at least two implant templates differ. In particular, this can be achieved through different information applied to the respective implant templates, for example the two-dimensional implant view or further optional information.

Favorably, the at least two implant templates differ from one another in the size and/or shape of the depicted implant view. For example, the carrier element of the implant templates may be of the same size and also of the same shape. However, the two-dimensional implant views of the at least two implant templates differ in size and/or shape. A user can thus place the at least two implant templates one after the other against a body structure of a patient and thus determine which size and/or shape best fits the body structure, and then select the appropriate implant to be finally implanted. The at least two implant templates can be provided in a sterile manner together in a sterile packaging.

Further, it may be advantageous if the at least two implant templates differ from one another in the size of the carrier element. In particular, identical two-dimensional implant views can thus be displayed on carrier elements of different sizes.

Furthermore, it may be advantageous if the at least two implant templates of the implant template system are formed in one piece. In particular, different views of an implant or implants of different sizes with identical implant views can thus be shown on the two implant templates of the implant template system. The one-piece configuration ensures, in particular, that all the required sizes of implants are actually provided to a user as corresponding implant views. For example, they can separate the implant templates from one another in the case of use, i.e. only once they are in an operating room.

Preferably, the implant template system is configured to be able to be cut to size and/or comprises predetermined breaking lines for separating the at least two implant templates of the implant template system formed in one piece with one another from one another. This design enables a user, in particular, to break down the implant template system in a simple and reliable manner into the individual implant templates.

Further, a method for determining an optimal implant size of an implant to be implanted in a human or animal patient is proposed, in which method one of the implant template systems described above is provided, wherein an implant template is selected from the implant template system, wherein the selected implant template is placed against a bone of the patient in such a way that the implant view of the implant on the implant template corresponds to the orientation of the implant to be implanted, wherein the size of the implant view depicted on the implant template is compared with the physiological situation of the patient, and wherein it is then decided whether the implant size depicted on the implant template is suitable for the patient or not. If the specific implant size fits, a user can select the corresponding implant and implant it into the body of the patient. If the implant size is not suitable, they can select a further implant template and perform the comparison again with the bodily structure, for example a bone or the like.

Preferably, the method is carried out until the most suitable implant size is determined. With such a method, in particular, optimally suitable implants can be determined without having to provide them in advance. Elaborately designed three-dimensional trial implants, which correspond in shape and size to the implants to be implanted, regardless of which material they are made of, for example a metal or a plastic material, are also not required. Instead, with the implant templates described above that can be formed in a simple and cost-effective manner, it is possible to determine an optimally suitable implant in a simple and reliable manner.

Furthermore, the use of one of the implant template systems described above is proposed for performing one of the methods described above.

### BRIEF DESCRIPTION OF THE DRAWINGS

The subsequent description of a preferred embodiment of the present disclosure serves in conjunction with the following drawings for further explanation.

FIG. 1 shows a schematic perspective partially broken view of an embodiment of an implant in the form of a bone plate, which is fixed to a spine with screws;

FIG. 2 shows a schematic perspective view of an embodiment of an implant template, which shows a two-dimensional view of the implant depicted in FIG. 1;

FIG. 3 shows a schematic view of the implant template from FIG. 2 when measuring on the body of the patient;

FIG. 4 shows a schematic depiction of an embodiment of an implant template system;

FIG. 5 shows a schematic perspective view of a further embodiment of an implant template;

FIG. 6 shows a schematic depiction of a portion of the embodiment of an implant template system depicted in FIG. 4;

FIG. 7 shows a schematic depiction of a further embodiment of an implant template system;

FIG. 8 shows a schematic depiction of a further embodiment of an implant template;

FIG. 9 shows a view of the implant template from FIG. 8 in the direction of arrow A when bending said implant template;

FIG. 10 shows a view of the implant template from FIG. 8 in the direction of arrow B when bending said implant template;

FIG. 11 shows a schematic depiction of a further embodiment of an implant template system;

FIG. 12 shows a schematic depiction of a further embodiment of an implant template;

FIG. 13 shows a view in the direction of arrow C of the implant template from FIG. 12 with a bent holding element;

FIG. 14 shows a view similar to FIG. 13, but with a deformed carrier element;

FIG. 15 shows a view of the implant template from FIG. 12 in the direction of arrow B in FIG. 12 after bending said implant template;

FIG. 16 shows a side view of an embodiment of a knee joint endoprosthesis;

FIG. 17 shows top view of a tibial component of the knee joint endoprosthesis depicted in FIG. 16;

FIG. 18 shows a schematic depiction of a further embodiment of an implant template system;

FIG. 19 shows a perspective view of a femoral component of a knee joint endoprosthesis;

FIG. 20 shows a further embodiment of an implant template with two two-dimensional implant views;

FIG. 21 shows a perspective depiction of an embodiment of a meniscal component of a knee joint endoprosthesis;

FIG. 22 shows a schematic depiction of a further embodiment of an implant template comprising two two-dimensional implant views;

FIG. 23 shows a schematic perspective view of an embodiment of an intervertebral disc implant;

FIG. 24 shows a schematic depiction of a further embodiment of an implant template system;

FIG. 25 shows a schematic depiction of a further embodiment of an implant template system; and

FIG. 26 shows a purely schematic depiction of an embodiment of an implant template system.

### DETAILED DESCRIPTION

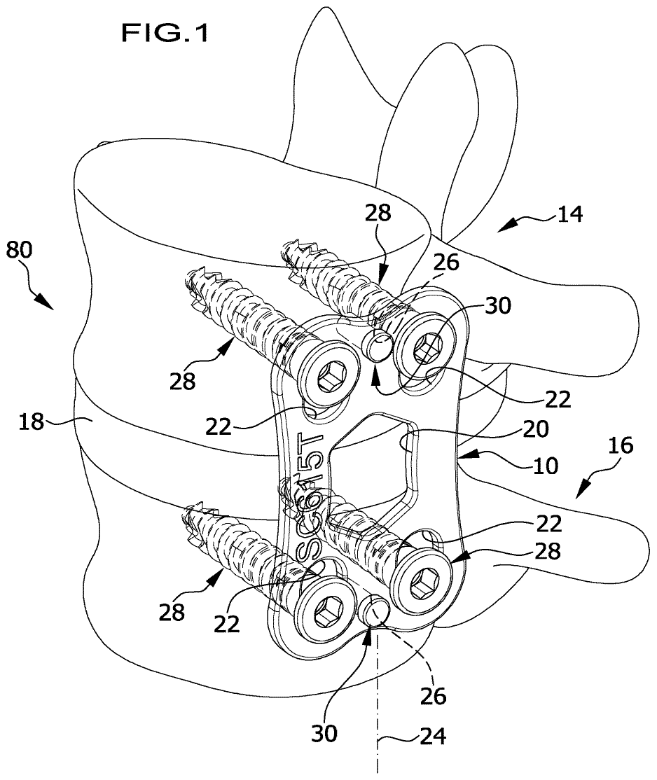

A first embodiment of an implant 10 is schematically depicted in FIG. 1. It is configured in the form of a bone plate 12, with which two adjacent vertebrae 14 and 16 of a spine 80, which are connected to one another by way of an intervertebral disc 18, are coupled to one another.

The bone plate 12 comprises a central perforation 20 and four screw holes 22 and defines a longitudinal axis 24. It is also configured symmetrically relative to a symmetry plane containing the longitudinal axis 24.

The screw holes 22 are shaped like elongate holes and extend in parallel with the longitudinal axis 24. The screw holes 22 are formed in the region of the four corners of the substantially rectangular bone plate 12. The perforation 20 is formed centrally on the bone plate 12 and has a substantially hexagonal contour. Outer corners of the bone plate 12 are rounded.

Lying on the longitudinal axis 24, two fixation holes 26 are formed on the bone plate 12, which are also designed as perforations of the bone plate 12. They have a circular cross-section. The two fixation holes 26 are each formed in the region between two adjacent screw holes 22.

The bone plate 12 is anchored with four bone screws 28 in the two vertebrae 14 and 16. Before anchoring with the four bone screws 28, the bone plate 12 is prefixed on the two vertebrae 14 and 16 with two bone pins 30 passing through the fixation holes 26.

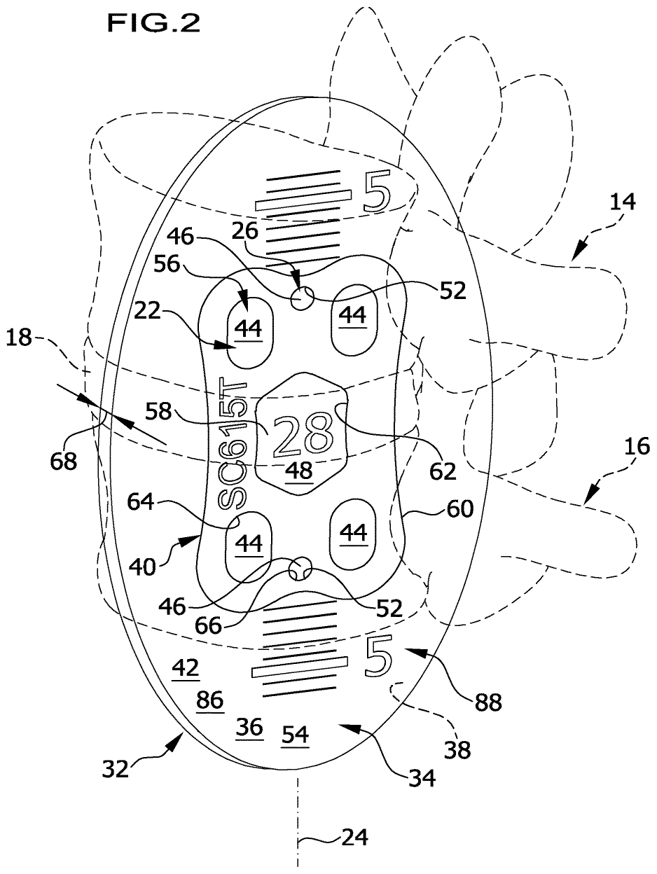

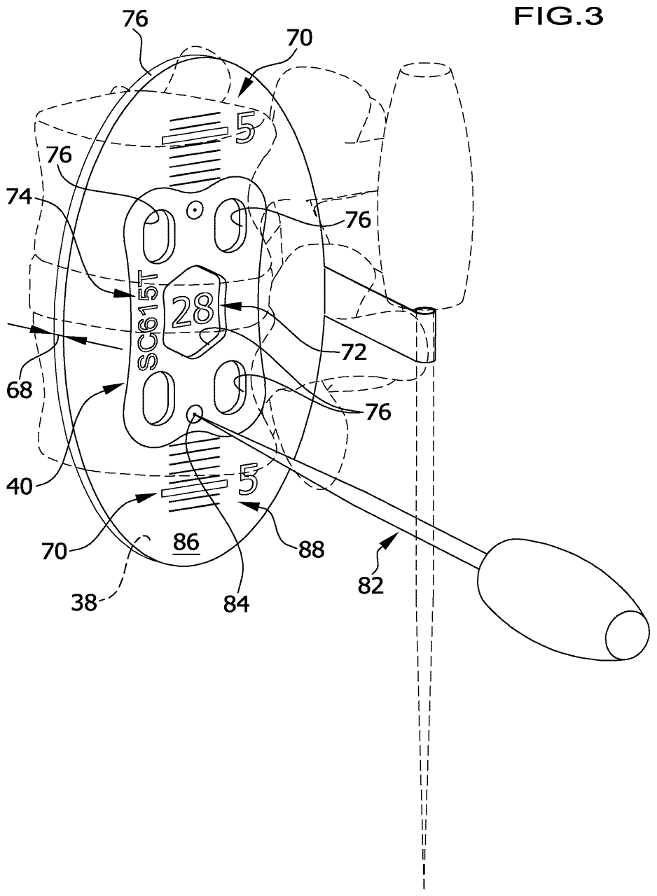

In order to determine the appropriate size of the implant 10, at least one implant template 32 is provided. FIGS. 2 and 3 show an embodiment of such an implant template 32.

The implant template 32 comprises a carrier element 34 with a front side 36 and a rear side 38. The carrier element 34 is formed from a flat material.

The carrier element 34 carries at least one two-dimensional implant view 40 of an implant 10 or has such a two-dimensional implant view 40.

Furthermore, the carrier element 34 has at least one see-through region 42, 44, 46, 48, which makes it possible to see through the carrier element 34.

The carrier element 34 is of plate-shaped or disc-shaped configuration. The embodiment depicted in FIGS. 2 and 3 shows a carrier element 34 that is of oval configuration.

Alternative embodiments of carrier elements 34 that are not depicted are of circular or polygonal configuration, in particular triangular, quadrangular, pentagonal, or hexagonal configuration.

The at least one see-through region 42, 44, 46 or 48 is configured in the form of a perforation 52 or in the form of a translucent or transparent carrier element region 54, 56, 58.

The implant view 40 defines an outer contour 60 and at least one inner contour 62, 64, and 66. The see-through region 42 is configured adjoining the outer contour 60. The see-through region 44 is configured adjoining the inner contour 64. The see-through region 46 is configured adjoining the inner contour 66. The see-through region 48 is configured adjoining the inner contour 62.

The implant view 40 comprises at least one implant perforation 20, 22, 26 of the implant 10. Thus, the embodiment of the implant template 32 shows four screw holes 22, two fixation holes 26, and a central perforation 20.

In order to establish the relationship of the implant openings 20, 22, 26 to the implant 10, which is schematically illustrated in FIG. 1, the same reference numerals are used to denote the implant perforations 20, 22, 26 in the embodiment of FIGS. 2 and 3 as in the case of the implant 10 depicted in FIG. 1.

The implant perforations 20, 22, 26, namely the four screw holes 22, the perforation 20, and the two fixation holes 26, each form one of the see-through regions 44, 46, and 48.

The carrier element 34 has a thickness 68, which is defined by a distance between the front side 36 and the rear side 38. The thickness 68 has a value of at most 3 mm. In particular, it is at most 1 mm.

Moreover, the thickness is at least 0.05 mm. In preferred embodiments, the thickness 68 is at least 0.5 mm.

The implant template 32 is made of one or more biocompatible materials. The carrier element 34 is made of a biocompatible carrier element material.

The carrier element 34 is further made of a plastic material. In the embodiment shown in FIGS. 2 and 3, it is polymethyl methacrylate (PMMA). In alternative embodiments, the plastic material is polyethylene terephthalate glycol (PETG) and/or polypropylene (PP).

The carrier element 34 is optionally made of a transparent and/or translucent carrier element material.

The implant view 40 is configured in the form of a top view of a side of the implant 10. In the embodiment of FIGS. 2 and 3, a top view of the bone plate depicted in FIG. 1 is shown on the front side 36 of the carrier element 34.

The implant view 40 is printed on the carrier element 34. In alternative embodiments, the implant view 40 is engraved or formed by laser marking or by a thin metallic stamped part.

In alternative embodiments, the implant view 40 is formed or applied on the rear side 38 of the carrier element 34.

In embodiments, the rear side 38 of the carrier element 34 is translucently matted. In this way, the see-through regions 42, 44, 46, and 48 are also configured in a translucent form. If in certain embodiments the matting is not realized in all regions of an otherwise transparent carrier element 34, individual see-through regions 42, 44, 46, 48 may also be configured to be transparent if the carrier element 34 is not translucently matted in this region.

In one embodiment, the carrier element 34 is of flexibly deformable configuration.

In a further embodiment, the carrier element 34 is of curved configuration corresponding to the depicted implant 10.

In one embodiment, the carrier element 34 is of plastically deformable configuration. Thus, the carrier element 34 can be bent into a desired shape by a user. Due to the plastic deformability of the carrier element 34, it then retains the deformation specified by the user.

The embodiment of FIGS. 2 and 3 has an optional measuring scale 70. In this embodiment, it is designed in the form of a millimeter scale. In the embodiment of FIGS. 2 and 3, the measuring scale 70 adjoins in the extension of narrow sides of the implant view 40 along a longitudinal axis 24, which forms a symmetry axis of the implant template 32. The measuring scale 70 is optionally printed, engraved, or formed by laser marking or by a thin metal stamped part.

The implant template 32 is configured to be sterilizable. It can be sterilized by being subjected to a gas and/or electromagnetic radiation and/or particle radiation and/or superheated steam.

In the embodiment of FIGS. 2 and 3, the carrier element 34 carries at least one further piece of implant-specific information. The implant-specific information is a size 72, which in the case of the embodiment of FIGS. 2 and 3 corresponds to a length that is denoted in FIGS. 2 and 3 with “28”. A further piece of implant-specific information is an article number of the depicted implant 10, indicated in the Figures with the specification “SC615T”.

In order to protect the implant-specific information, thus in particular the implant view 40, from abrasion, the implant template 32 has a protective layer not shown in the Figures, which completely covers the information applied to the carrier element 34, in particular the at least one implant view 40 and as described optional further information.

In one embodiment, the protective layer is configured in the form of a transparent or translucent adhesive strip. In this way, a transparent or at least translucent property of the carrier element 34 is obtained or predetermined. A translucent adhesive strip can thus take on the function of a translucent matting. For example, a transparent carrier element 34 may be coated with a translucent adhesive strip, so that in the event that the protective layer completely covers a front side 36 or a rear side 38, regions of the implant template that are not covered by the implant view 40 or implant-specific information remain translucent.

Edges 76 of the carrier element 34, which may delimit an outer contour of the carrier element 34 and optionally perforations thereof, are rounded in a manner not depicted in more detail or have a chamfer to prevent injury to body tissue when using the implant template, in particular upon insertion thereof into a body of the patient.

The implant template 32 is configured as a single-use article. This means that after being used once on a patient, it is not reprocessed and sterilized, but disposed of directly.

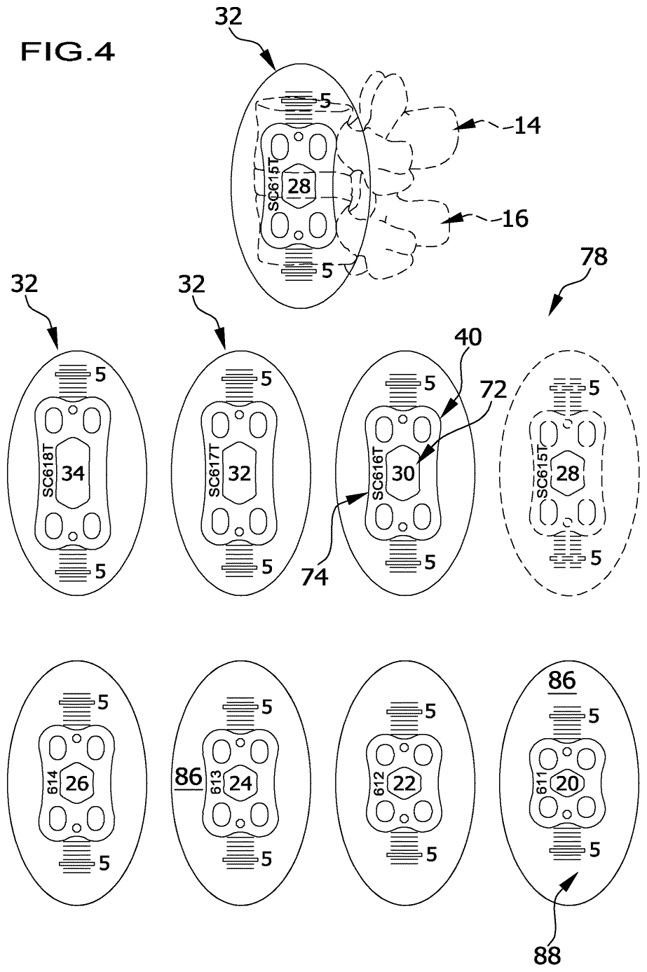

An implant template system 78 is depicted as an example in FIG. 4. It comprises at least two implant templates 32. The arrangement from FIGS. 2 and 3 is schematically depicted in the upper region of FIG. 4. The implant template 32 brought up to the two vertebral bodies 14, 16 is shown with dashed lines as part of the implant template system 78.

The implant template system 78 of the embodiment depicted in FIG. 4 comprises a total of eight implant templates 32, all of which are designed differently. Thus, the at least two implant templates 32 differ from one another. In the embodiment of the implant template system 78 of FIG. 4, the implant templates 32 differ from one another in the size of the implant view 40 depicted. This can be seen in particular directly on the basis of the size of the implant view 40, in addition to the different specification of the size 72 and the different specification of the article number 74.

The implant template system 78 thus comprises eight implant templates 32, which show eight implants 10 of different sizes in the form of bone plates 12. A length of the respective bone plate 12 varies in a grid of 2 mm from a minimum length of 20 mm to a maximum length of 34 mm.

The carrier elements 34 of the implant templates 32 of the implant template system 78 of FIG. 4 are of oval configuration and are all of identically sized configuration.

With the implant template system 78, a method for determining an optimal implant size of an implant 10 to be implanted in a human or animal patient can be carried out. For this purpose, first an implant template system 78 is provided.

To determine the size of the implant 10 ultimately to be implanted, which is to remain permanently in the patient, one of the implant templates 32 of the implant template system 78 is selected and brought laterally up to the body structure, in FIGS. 2 to 4 the body structure being schematically represented by a section of a spine 80, such that a user sees the implant view 40 of the implant template 32 from the side from which the implant 10 is to be fixed to the body structure. The implant template 32 is brought up to the body structure, i.e. for example the spine 80 of the patient, in such a way that the implant view 40 of the implant 10 on the implant template 32 corresponds to the orientation of the implant to be implanted.

A user can now compare the size of the implant view 40 shown on the implant template 32 with the physiological situation, i.e. the body structure, of the patient and then decide whether the implant size shown on the implant template 32 is suitable for the patient or not. If it is not suitable, they then place the selected implant template 32 to the side and select one of the remaining implant templates 32 of the implant template system 78. They then carry out the method until the implant size that in their opinion is most suitable has been determined. They can gather this information directly from the implant template 32, namely in particular based on the specified size 72 or the specified article number 74.

In particular with bone plates 12, as described, it can be determined in a simple and cost-effective manner whether bone screws 78 are able to be reliably anchored in the vertebrae 14 and 16 considering the position of the screw holes 22.

When the implant template 32 with the implant 10 that is most suitable in size and optionally also in shape has been determined, for example, a position of the fixation holes 26 on the vertebrae 14 and 16 can be marked with a medical instrument 82 in the form of a center punch. To this end, the instrument 82 has a tip 84 with which the vertebrae 14 and 16 can be marked. At the marked points, the bone pins 30 described in connection with FIG. 1 can be inserted, such that the implant 10 corresponding to the implant view 40 can then be slid over the bone pins 30 and brought up to the vertebrae 14 and 16. In a final step, the bone plate 12 can then be fixed to the vertebrae 14 and 16, for example with the four bone screws 28 depicted in FIG. 1.

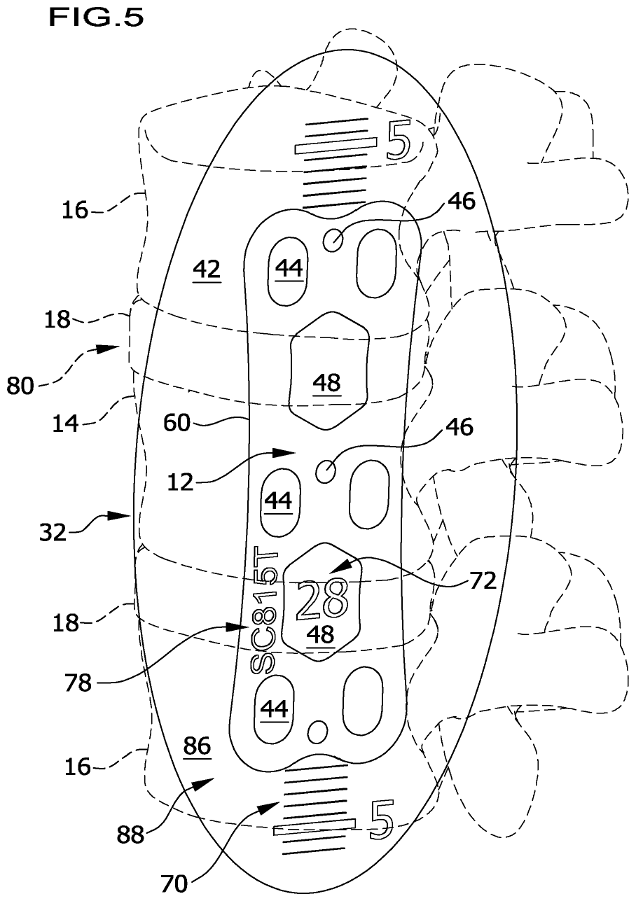

In the following, further embodiments of implant templates 32 and implant template systems 78 are described schematically in connection with FIGS. 5 to 26. The properties of the implant templates 32 of these embodiments correspond to those explained above in connection with the embodiments in FIGS. 2 to 4.

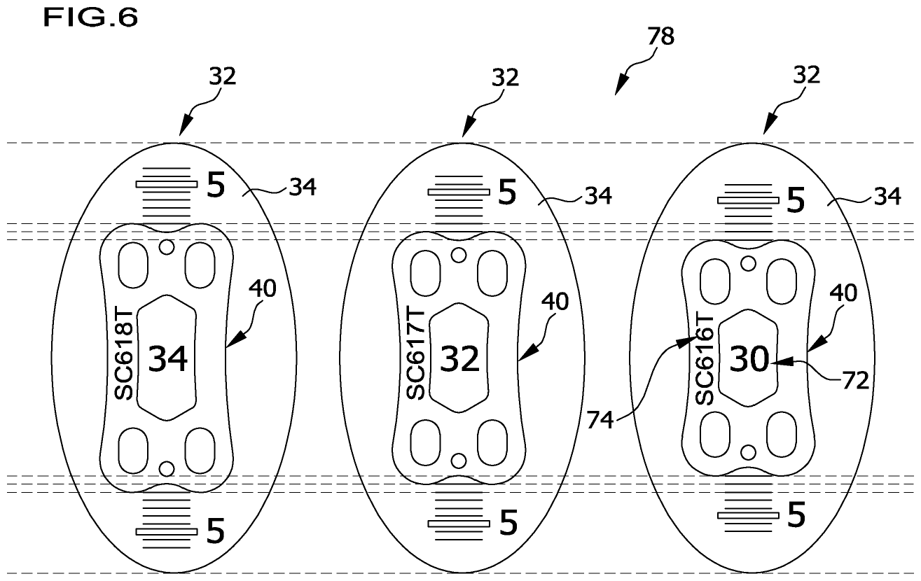

To denote identical or similar components or elements, the same reference numerals are used in the embodiment of FIG. 5 as in the embodiment of FIGS. 2 to 4. This also applies accordingly to the further embodiments depicted in FIGS. 6 to 26.

FIG. 5 shows an embodiment of an implant template 32, which shows a bone plate 12 that is provided to connect a total of three vertebrae 14 and 16 in order to stiffen two movement segments of a spine 80 that are formed by two intervertebral discs 18 between in each case two adjacent vertebrae 14 and 16 respectively.

Accordingly, the implant view 40 shows two see-through regions 48, each corresponding to a perforation 20 on the implant 10.

The implant templates 32 of the embodiments depicted in FIGS. 2 to 5 are designed in such a way that the carrier element 34 projects beyond the implant view 40 on all sides on the outer contour 60. The implant template 32 thus comprises a carrier element region 86 without an implant 40, which surrounds the implant view. The carrier element region 86 may serve, in particular, for handling the implant templates 32, namely in the form of a holding element 88. Thus, the implant template 32 can be gripped by a user with two fingers or a suitable holding instrument, for example a needle holder or forceps, and brought up to a bodily structure of the patient, for example their spine 80, as shown schematically in FIGS. 2 to 5.

FIG. 6 shows schematically an embodiment of an implant template system 78 comprising three implant templates 32. The carrier elements 34 thereof are identically shaped. In contrast, the depicted implant views 40 are of different sizes. This is shown by the size of the implant view 40 and the size 72 indicated in each case and the article number 74 indicated in each case.

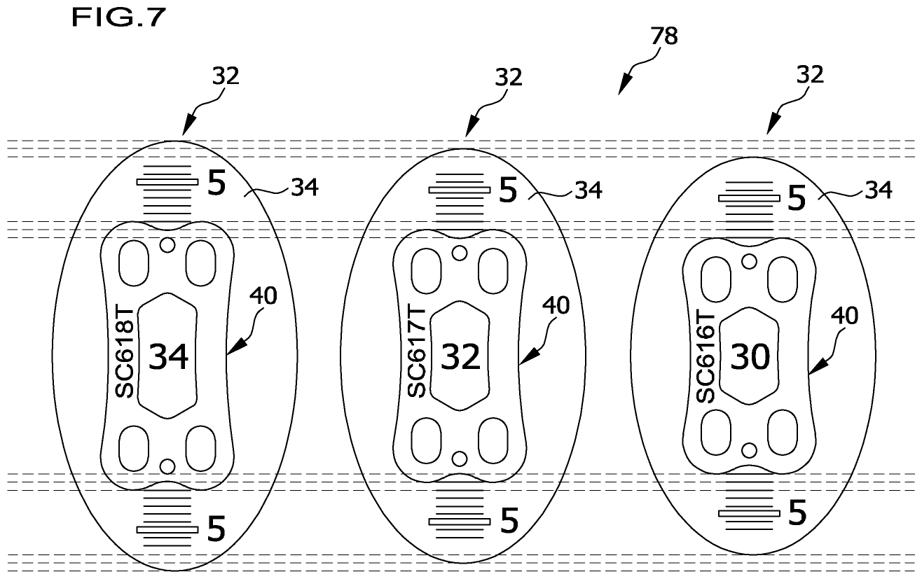

FIG. 7 shows a further embodiment of an implant template system 78. In this implant template system 78, the carrier elements 34 are geometrically similar, but are of differently sized configuration. Thus, the at least two implant templates 32 differ from one another in the size of the carrier element 34.

In the implant template system 78, the implant views 40 are also configured differently. They correspond to the three implant views 40 of the three implant templates 32 depicted in FIG. 6. Thus, the implant templates 32 of the implant template system 78 also differ from one another in the size of the depicted implant views 40.

FIG. 8 shows an embodiment of an implant template 32 in a top view. Side views of said implant template 32 are schematically depicted in FIGS. 9 and 10. FIGS. 9 and 10 show as an example how the carrier element 34 can be deformed, in particular bent. In particular, from a planar starting position, which is shown with dashed lines in FIGS. 9 and 10, the carrier element 34 can be bent, in particular relative to semi-axes, which extend in parallel and perpendicularly to the longitudinal axis 24 of the depicted implant 10. A deformation may optionally be carried out in such a way that the carrier element 34 is concavely or convexly curved in the direction toward a body structure.

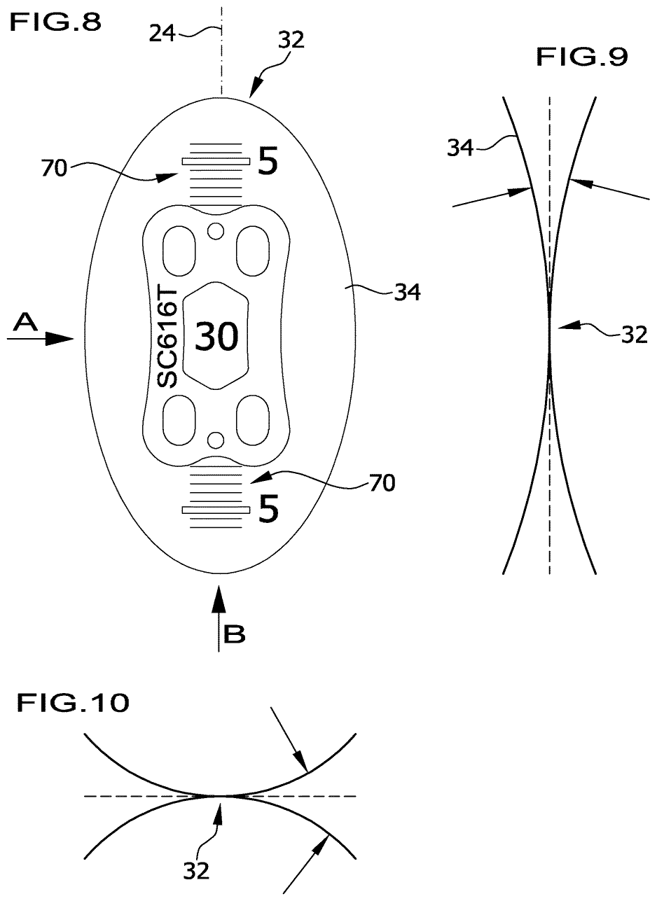

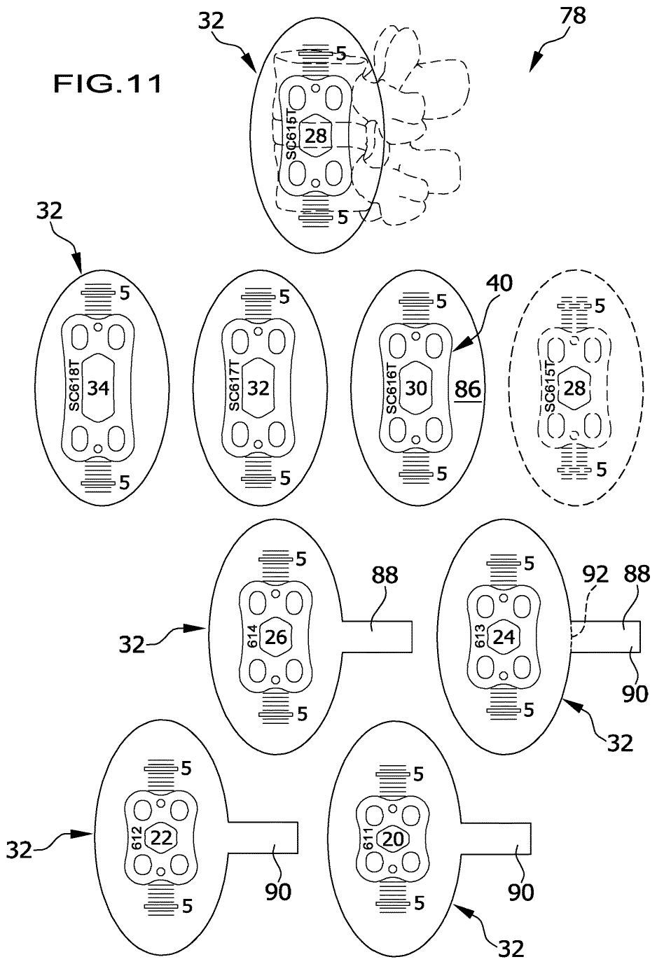

A further embodiment of an implant template system 78 is depicted schematically in FIG. 11. Four of the eight implant templates, which have a total of eight different-sized implant views 40, are equipped with a holding element 88 for holding the implant template 32, in particular with a holding instrument not shown in the Figures, namely in the form of a holding element 88 configured projecting from the carrier element 34. Said holding element 88 is formed in one piece with the carrier element 34, namely from the flat material from which the carrier element 34 is formed, for example by cutting out or punching out.

In the case of the four implant templates 32 depicted in FIG. 11 in the lower region, the holding element 88 in each case is configured in the form of a tab 90 projecting from the carrier element 34 in the direction of a semiminor axis of the oval carrier element 34.

The tab 90, thus the holding element 88, is configured to be deformable, namely bendable, along a bending line drawn with dashed lines in FIG. 11 in one embodiment of an implant template 32. This makes it possible to transfer the holding element 88 and the carrier element 34 into a handling position such that in said position they enclose a holding angle 94, which is in a range between 0° to about 120°. In particular, the holding angle 94 may be 0°.

In addition, the carrier element 34 forms further holding elements by way of carrier element regions 86 without an implant view 40. Thus, a user has several options to hold the implant template 32 and to bring it up to a body structure of a patient.

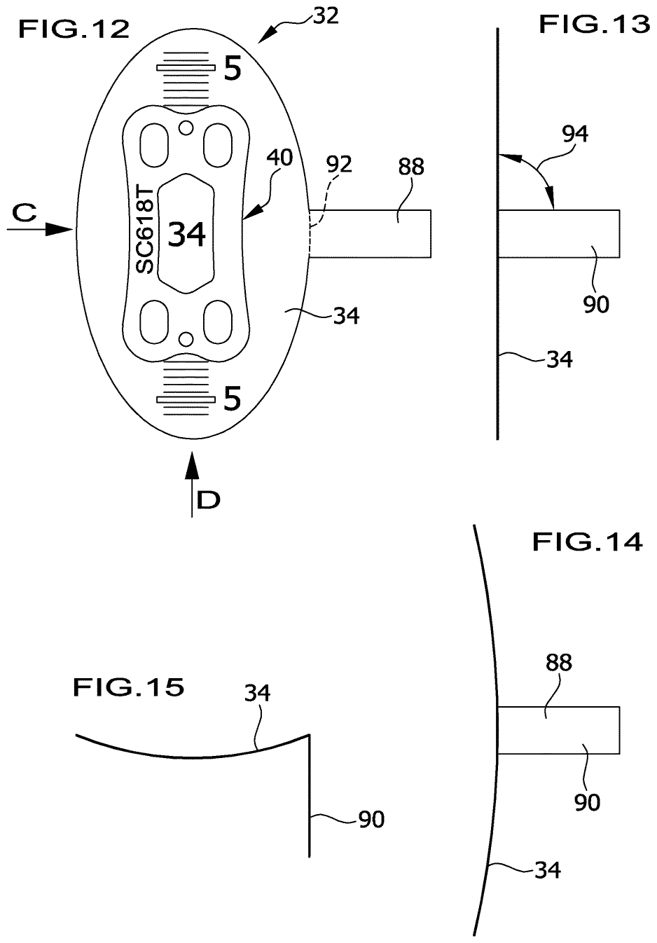

FIGS. 12 to 14 show a further embodiment of an implant template 32. The implant template 32 is schematically depicted in FIG. 12 in a starting position in which the carrier element 34 and the laterally projecting holding element 88 lie in a plane. In particular, it is a plane defined by the flat material from which the carrier element 34 is formed.

A schematic side view of the implant template 32 is depicted in FIG. 13. Here, the tab 90 is bent along the bending line 92 by the holding angle 94. The holding angle 94 is in a range between 0° and about 120°. In FIG. 13, it is about 90°. FIG. 13 schematically shows the carrier element 34 undeformed.

In FIG. 14, the implant template 34 from FIG. 13 is depicted with a carrier element 34 bent relative to a semiminor axis of the oval carrier element 34. FIG. 15 in turn shows a deformed carrier element 34 relative to a semimajor axis of the oval carrier element 34.

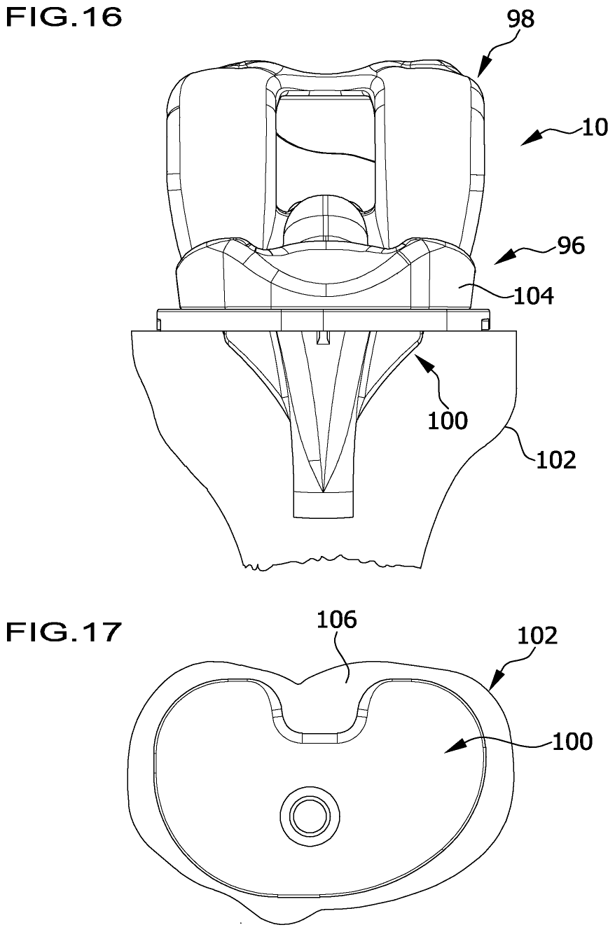

FIG. 16 schematically shows a further embodiment of an implant 10 in the form of a knee joint endoprosthesis 96 comprising a femoral component 98 for fixing to a femur of a patient, which is not depicted in the Figures, a tibial component 100 for anchoring to a tibia 102 of a patient, and a meniscal component 104 arranged between the tibial component 100 and the femoral component 98.

Before the implantation of the knee joint endoprosthesis 96, the tibia 102 is prepared and a planar tibial face 106 is formed by a saw cut.

FIG. 17 schematically shows a top view of a tibial component 100 anchored to the tibia 102.

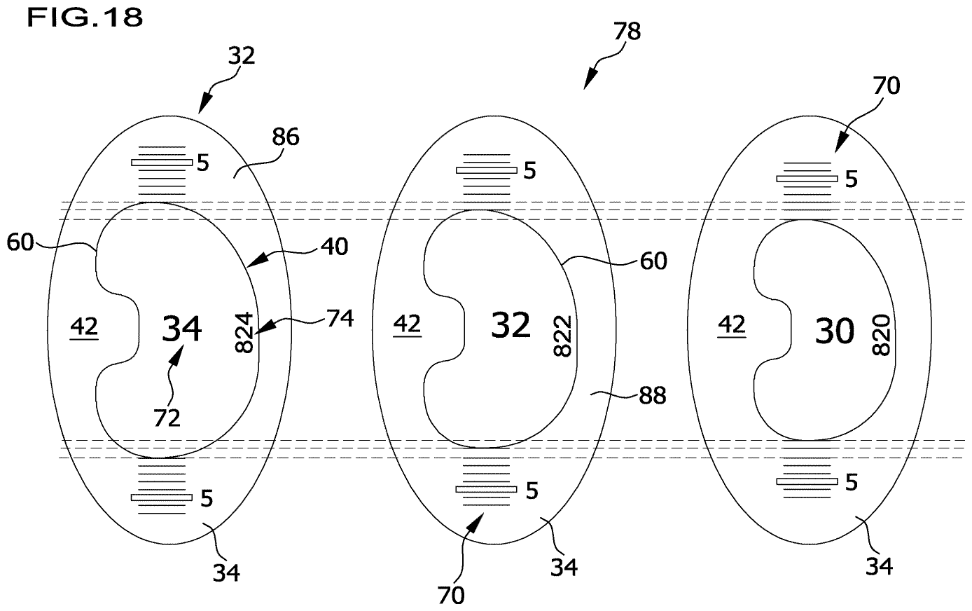

In order to determine the tibial component 100 that is optimally suitable for a patient, for example, an embodiment of an implant template system 78 that is schematically depicted in FIG. 18 is provided. This implant template system 78 comprises three implant templates 32, which each show an implant view 40 of the tibial component 100, namely a top view thereof from above. The carrier elements 34 are each larger than the implant view, such that a see-through region 42 adjoining the outer contour 60 of the implant view 40 is formed. Said see-through region can simultaneously form a carrier element region 86 that can be used as a holding element 88. Optional measuring scales 70 are also depicted in the case of these implant templates, too.

In this embodiment of the implant template system 78, the carrier elements 34 are of identically sized configuration, but the implant views 40 differ in their size. This can be seen directly visually on the one hand, and other hand due to the specified additional information, as there is the size 72 as well as the article number 74 of the implant 10, the implant view 40 of which is formed on the carrier element 34.

With the implant template system 78, the optimally fitting size of the implant 10, thus the tibial component 100, can be determined in the manner described above by comparing the bodily structure with the implant view 40 depicted on the implant templates 32.

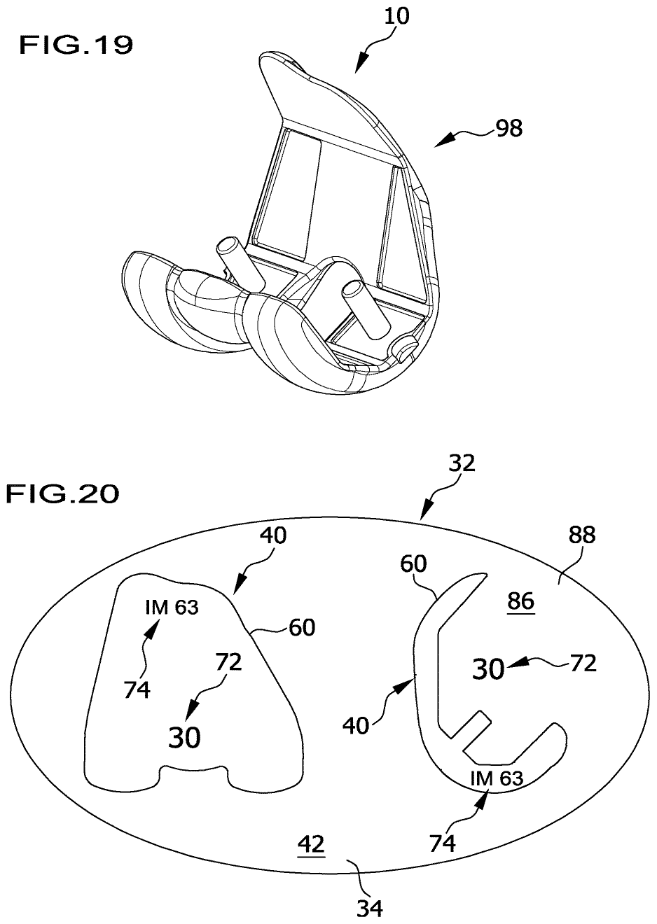

FIG. 19 schematically shows a perspective view of an embodiment of a femoral component 98.

In order to determine the size of the femoral component to be implanted, implant templates 32 can be used, which are shown in FIG. 22 as an example. Here, two different implant views 40 of the implant 10, which is formed by the femoral component 98, are schematically depicted on the carrier element 34. These are two mutually perpendicular views, namely one of the femoral component 98 from the front, depicted on the left on the carrier element 34 in FIG. 20, and a view of the femoral component 98 from the side, depicted in the right region of the carrier element 34 in FIG. 20.

With the implant template 32, which is shown as an example in FIG. 20, the size of the femoral component 98 can be determined, namely by the left region of the implant template 32 with the implant view 40 being brought up to the knee joint from the front. In addition, the side view of the femoral component 98 on the implant template 32 can be brought up to the knee joint from the side. A holding element 88 is defined by a carrier element region 86 without an implant view 40. This carrier element region 86 adjoins in each case outer contours 60 of the implant views 40.

In this embodiment, too, additional information specific to the implant 10 is optionally displayed, namely, in particular, a size 72 and an article number 74 of the implant 10, the implant views 40 of which are depicted on the carrier element 34.

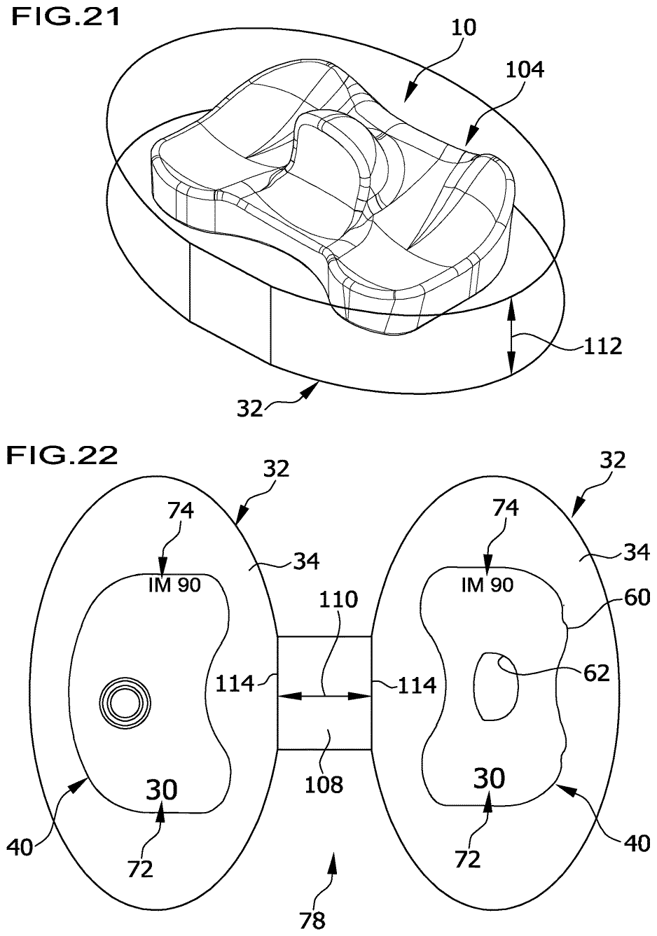

FIG. 21 schematically shows a meniscal component 104, which forms an implant 10. Further, an implant template 32 is schematically depicted in FIG. 21. The implant template 32 basically forms an implant template system 78 comprising two implant templates 32 with oval carrier elements 34, which are connected by way of a tab-shaped connecting element 108. A distance 110 defined by the connecting element 108 between the two carrier elements 34 corresponds to a height 112 of the meniscal component 104.

Connecting lines 114 between the connecting element 108 and the carrier elements 34 may also be used as bending lines or as separating lines or predetermined breaking points. For example, the connecting lines 114 can be used as bending lines in order to orient the two carrier elements 34 in parallel with one another as shown schematically in FIG. 21, so that the two carrier elements 34 are each bent by 90° relative to the connecting element 108.

Alternatively, the two carrier elements 34 can also be separated from the connecting element 108 along the connecting lines 114, such that two separate implant templates 32 are formed, which together form an implant template system 78.

Thus, the implant template system 78 is formed in one piece and comprises two implant templates 32.

The carrier elements 34 are each configured identically. On the other hand, the implant views 40 differ from one another in their shape. The size is identical, as can be read from the implant-specific information size 72 and article number 74 on the implant templates 32. One of the implant templates 32 shows the implant 10, i.e., the meniscal component 104, in a top view from below, namely in FIG. 22 on the left, and in a top view from above, in FIG. 22 on the right.

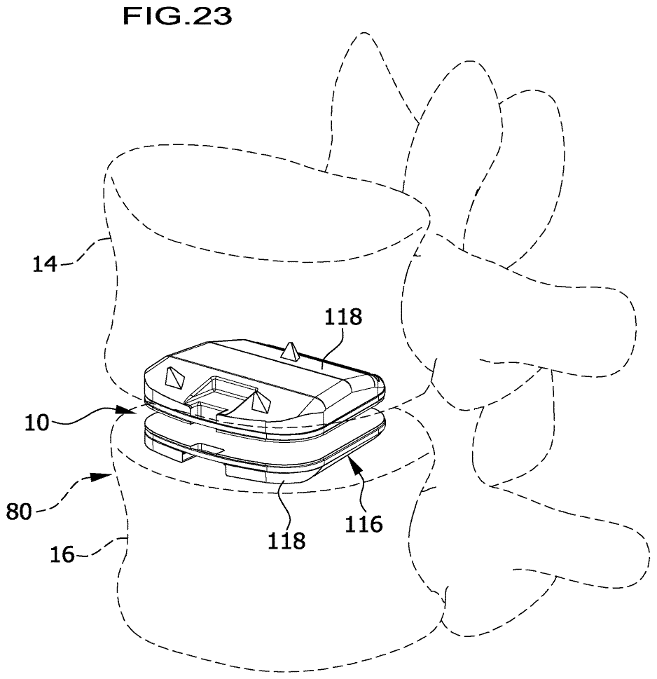

Schematically depicted in FIG. 23 is a spine 80 with an implant 10, inserted in an intermediate space between two vertebrae 14 and 16, in the form of an intervertebral disc implant 116. If the intervertebral disc implant 116 is configured having two abutment plates 118 that are movable relative to one another, it can be used as an intervertebral disc implant 116. If the abutment plates 118 are configured to be immovable relative to one another, the implant 10 forms an intervertebral implant, for example for stiffening a movement segment of the spine 80, which in the spine is originally formed by an intervertebral disc 18 between the vertebrae 14 and 16. When the intervertebral disc 18 is removed, an intervertebral disc implant 116 or an intervertebral implant can be inserted into the vacated intervertebral disc space between the vertebrae 14 and 16.

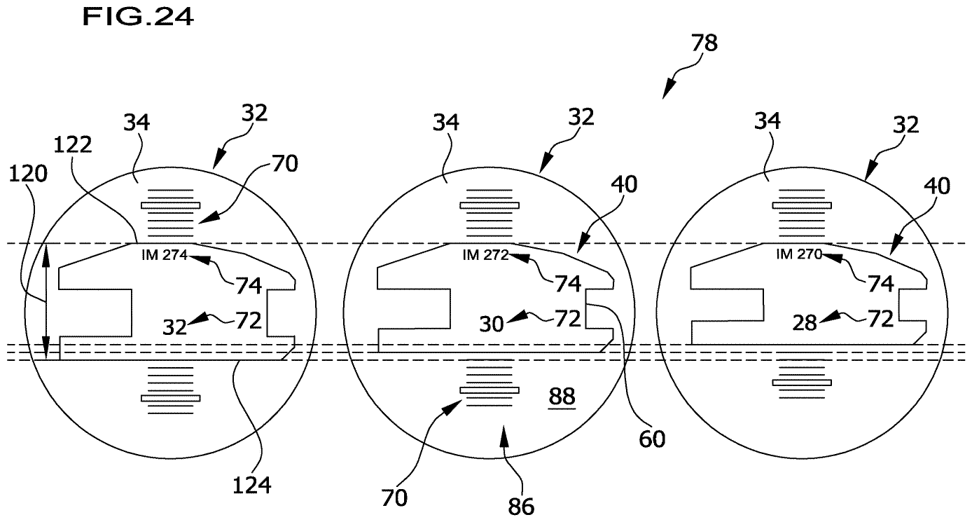

FIG. 24 schematically shows a further embodiment of an implant template system 18. Three implant templates 32 are shown here as an example. The carrier elements 34 are of circular configuration and each carry an implant view 40 in the form of a lateral top view of the intervertebral disc implant 116 from FIG. 23.

The implant views 40 of the three implant templates 32 differ from one another in their size. This is directly recognizable by the implant views 40 themselves and by the implant-specific information indicated on the implant templates 32, namely size 72 and article number 74.

Measuring scales 40 serve to aid a user in determining the optimal size of the implant 10 to be implanted by, as already described, one of the implant templates 32 being selected and brought up to the spine 80 from the side in order to determine a height of the intervertebral disc space, thus a distance of the two vertebrae 14 and 16 from one another. This distance ideally corresponds to a height 120 of the intervertebral disc implant, thus a distance between a top side 122 and a bottom side 124 of the implant 10 shown as an implant view 40. Thus, with the implant template system 78, a height of the intervertebral disc space and thus the appropriate implant 10 can be determined in a simple manner.

As already explained in the case of other embodiments, the implant templates 32 can be handled by hand or with holding instruments by being gripped and handled at a carrier element region 86 without an implant view 40, which thus forms a holding element 88.

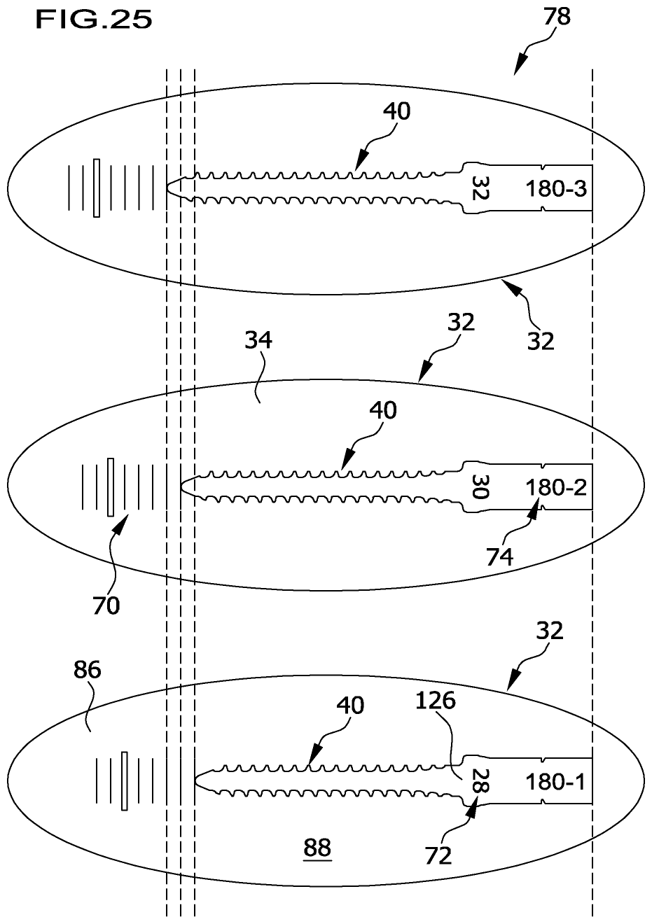

A further embodiment of an implant template system 78 is depicted schematically in FIG. 25. As an example, it comprises three implant templates 32, which each comprise carrier elements 34 of identical size and oval shape. Each carrier element 34 has an implant view 40 of an implant 10, which is configured in the form of a bone screw configured as a pedicle screw.

The implant views 40 differ in their size, which is directly recognizable and indirectly readable on the optional implant-specific information, namely size 72 and article number 74.

For use, as usual, one of the implant templates 32 of the implant template system 78 is selected and brought up to a bodily structure of the patient, namely a pedicle of a vertebra, in order to determine a maximum possible length of the bone screw 126 to be inserted by comparison with the implant view 40 depicted on the implant template 32.

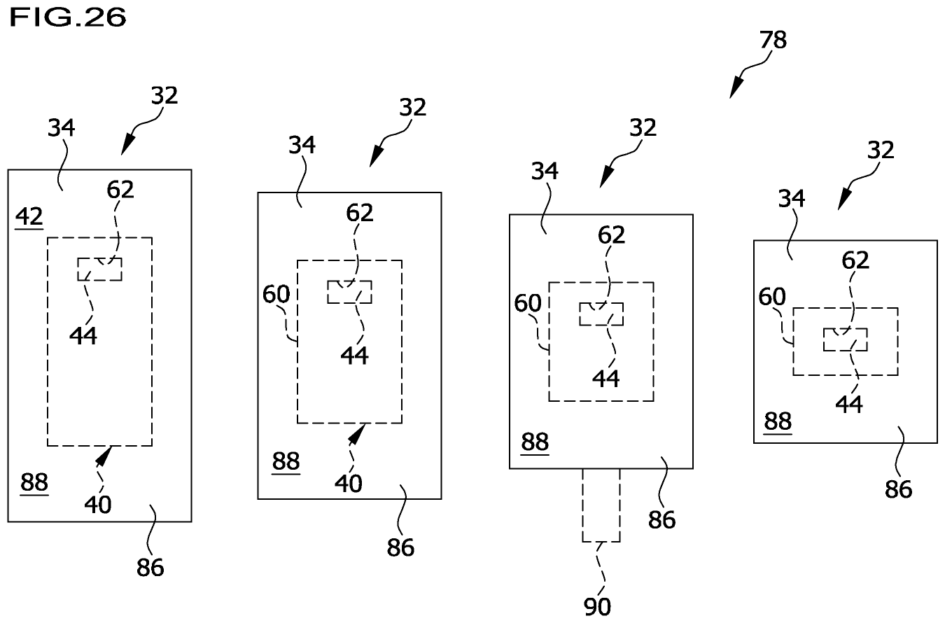

Finally, FIG. 26 shows schematically and in a maximally abstract way an embodiment of an implant template system 78 with at least two implant templates 32. The carrier elements 34 of the implant templates 32 carry or have an implant view 40 of an implant 10 to be implanted into the body of a patient. The implant view 40 has a see-through region 42 adjoining the outer contour 60 of the implant view 40 and a see-through region 44 adjoining an inner contour 62 of the implant view 40.

The implant views 40 of the implant templates 32 differ both in size and shape. Further, the carrier elements 34 are of different sizes. Carrier element regions 86 without an implant view 40 form holding elements 88 for handling the implant templates 32 and bringing them up to a body structure of the patient that is to be replaced in whole or in part by the implant 10, the implant view 40 of which is depicted on the implant templates 32.

The carrier elements 34 optionally have the properties described above in connection with the already described embodiments, such as elastic or plastic deformability of the carrier element 40. Not shown in FIG. 26 are further implant-specific information such as size 72 or article number 74 or one or more measuring scales that can be used for determining the size and for selecting the implant 10 to finally be implanted permanently.

In the implant template system 78 depicted in FIG. 26, the implant view 40 may show any implant 10 in a top view from any side. The implant 10 may be a spinal implant, a bone plate, a bone screw, an intervertebral disc implant, an intervertebral implant, a knee joint implant, a tibial plate of a knee joint implant, or a femoral component of a knee joint implant, or a meniscal component of a knee joint implant.

The implant templates 32 of the implant template system 78 can either be formed separately from one another or may also be formed in one piece. In the latter case, they are optionally configured to be cut to size or comprise predetermined breaking lines for separating the at least two implant templates 32 from one another.

Furthermore, optionally, holding elements 88 in the form of tabs 90 may also be formed on the carrier elements 34, as shown schematically with dashed lines on one of the implant templates 32 in FIG. 26.

As described above in conjunction with preferred embodiments, with implant templates 32, in particular, shape and/or size of an implant to be finally implanted in a patient can be determined by a surgeon in a simple and cost-effective manner. The implant templates 32, preferably made of a plastic material, enable a direct comparison of a size of the implant view 40 depicted on the implant template 32 with a body structure.

If the carrier element 34 is configured to be completely or partially translucent in regions without an implant view 40, it can thus be directly determined by a user whether the implant template 32 has been brought sufficiently close to the bodily structure of the patient, for example a bone structure such as the spine 80. The structure present behind the carrier element 34 becomes more concrete and more clearly visible the closer the implant template 32 is brought to the body structure. The optimal visibility is achieved when the implant template 32 abuts directly against the body structure. Thus, a translucent carrier element 34 in particular can also be used qualitatively for determining a distance of the implant template 32 from the body structure.

Unlike with known three-dimensional test trial implants that are used before an implant is implanted to determine an implant size, complex three-dimensional implant structures do not have to be created here. Rather, the proposed implant templates 32 utilize the fact that a surgeon typically sees the body structure from (only) one direction and with an implant template 32 that shows the implant 10 in a top view from exactly this direction, namely a corresponding implant view 40 on the carrier element 34, a direct size comparison is made possible.

In particular, such a size comparison is possible even when the body structures are not yet prepared for the insertion of the implant 10. For example, the size of the implant shown schematically in FIG. 23 can be determined before the intervertebral disc 18 between the vertebrae 14 and 16 is removed.

The implant templates 32 can be offered, in particular, individually packaged in a sterile manner. It is also possible to offer implant template systems 78 having two or more implant templates 32 in one single sterile packaging, such that after opening in a sterile environment, for example an operating room, all implant templates 32 of the implant template system 78 are available.

### LIST OF REFERENCE NUMERALS

10 implant
12 bone plate
14 vertebra
16 vertebra
18 intervertebral disc
20 perforation
22 screw hole
24 longitudinal axis
26 fixation hole
28 bone screw
30 bone pin
32 implant template
34 carrier element
36 front side
38 rear side
40 implant view
42 see-through region
44 see-through region
46 see-through region
48 see-through region
52 perforation
54 carrier element region
56 carrier element region
58 carrier element region
60 outer contour
62 inner contour
64 inner contour
66 inner contour
68 thickness
70 measuring scale
72 size
74 article number
76 edge
78 implant template system
80 spine
82 instrument
84 tip
86 carrier element region
88 holding element
90 tab
92 bending line
94 holding angle
96 knee joint endoprosthesis
98 femoral component
100 tibial component
102 tibia
104 meniscal component
106 tibial face
108 connecting element
110 distance
112 height
114 connecting line
116 intervertebral disc implant
118 abutment plate
120 height
122 top side
124 bottom side
126 bone screw

1. An implant template comprising a carrier element having a front side and a rear side,
the carrier element being formed from a flat material,
the carrier element carrying or having at least one two-dimensional implant view of an implant, and
the carrier element having at least one see-through region that allows a user to see through the carrier element.

2. The implant template according to claim 1, wherein the at least one see-through region comprises a perforation.

3. The implant template according to claim 1, wherein the at least one see-through region comprises a translucent or transparent carrier element region.

4. The implant template according to claim 1, wherein:
the at least one two-dimensional implant view defines an inner contour and/or an outer contour, and
the at least one see-through region is arranged or formed adjoining the inner contour and/or the outer contour.

5. The implant template according to claim 1, wherein the at least one two-dimensional implant view comprises at least one implant perforation of the implant.

6. The implant template according to claim 5, wherein the at least one implant perforation:
a) forms the at least one see-through region, and/or
b) comprises a screw hole or a fixation hole.

7. The implant template according to claim 1, wherein the carrier element is plate-shaped or disc-shaped.

8. The implant template according to claim 1, wherein:
the carrier element has a thickness defined by a distance between the front side and the rear side, and
the thickness has a value of at most 3 mm.

9. The implant template according to claim 1, wherein the carrier element is made of at least one of:
a) a biocompatible carrier element material,
b) a plastic material,
c) a transparent carrier element material, or
d) a translucent carrier element material.

10. The implant template according to claim 1, wherein the at least one two-dimensional implant view depicts a top view of a side of the implant.

11. The implant template according to claim 1, wherein the rear side of the carrier element is translucently matted.

12. The implant template according to claim 1, wherein the carrier element has at least one of:
a) a flexibly deformable configuration,
b) a curved configuration corresponding to the implant, and/or
c) a plastically deformable configuration.

13. The implant template according to claim 1, further comprising at least one holding element for holding the implant template.

14. The implant template according to claim 13, wherein the at least one holding element is at least one of:
a) a carrier element region without a two-dimensional implant view,
b) configured projecting from the carrier element,
c) formed in one piece with the carrier element,
d) configured as a tab projecting from the carrier element, or
e) deformable relative to the carrier element.

15. The implant template according to claim 13, wherein:
the at least one holding element and the carrier element in a handling position enclose a holding angle, and
the holding angle is in a range of 0°to about 120°.

16. The implant template according to claim 1, wherein the carrier element carries or comprises at least one of:
at least one further piece of implant-specific information, or
at least one measuring scale.

17. The implant template according to claim 1, wherein:
the implant template has a protective layer, and
the protective layer completely covers information applied to the carrier element.

18. The implant template according to claim 1, wherein the carrier element has two different implant views.

19. The implant template according to claim 1, wherein the implant depicted on the implant template is a spinal implant, a bone plate, a bone screw, an intervertebral disc implant, an intervertebral implant, a knee joint implant, a tibial plate of a knee joint implant, or a femoral component of a knee joint implant.

20. An implant template system comprising at least two implant templates according to claim 1, wherein the at least two implant templates differ from one another.
